(19) United States
(12) Patent Application Publication
(10) Pub. No.: US 2014/0275692 A1
(43) Pub. Date: Sep. 18, 2014

(54) MODIFIED SURFACE ENERGY NON-WOVEN FILTER ELEMENT

(71) Applicants: Shagufta Patel, Fort Worth, TX (US); John A. Krogue, Mineral Wells, TX (US)

(72) Inventors: Shagufta Patel, Fort Worth, TX (US); John A. Krogue, Mineral Wells, TX (US)

(21) Appl. No.: 14/212,774

(22) Filed: Mar. 14, 2014

Related U.S. Application Data
(60) Provisional application No. 61/798,735, filed on Mar. 15, 2013.

(51) Int. Cl. B01D 39/16; C07C 7/144; B01D 17/04

(52) U.S. Cl. CPC B01D 39/1623; B01D 17/045; C07C 7/144
USPC 585/818; 210/500.1; 210/497.1

(57) ABSTRACT

A non-woven low surface energy filter element designed to have improved removal of a dispersed liquid phase from a continuous liquid phase is disclosed.

### CROSS-REFERENCE TO RELATED PATENT APPLICATION

The present application claims the benefit of and priority to U.S. Provisional Application No. 61/798,735 filed on Mar. 15, 2013, which is incorporated herein by reference in its entirety.

### FIELD OF THE INVENTION

This invention generally relates to filters, and more specifically to low surface energy filter elements.

### BACKGROUND OF THE INVENTION

Spirally wound non-woven filter elements are known in the art. Recently, a helical wound tube of plural sheets made of at least one non-woven fabric of a homogeneous mixture of a base and a compressed binder material was disclosed as a filter element (U.S. Pat. No. 8,062,523). As described in the patent, each sheet is self-overlapped and compressed to overlap another sheet, and the individual sheets are selected to have different porosities and densities.

Similarly, U.S. Pat. No. 6,168,647 discloses a multi-stage vessel with a tubular separator/coalescer gas filter element disposed therein. The tubular separator/coalescer filter element has a filter wall and a hollow core, wherein the filter wall consists of multi-overlapped layers of non-woven fabric strips. The selected density and porosity of the separator/coalescer filter element prevents solids and pre-coalesced liquids from passing through the filter element and into a second stage of the multi-stage vessel. U.S. Pat. No. 5,893,956 also discloses a tubular filter element, wherein a roll of non-woven fabric strip is mounted on a roll support consisting of an upright member onto which are mounted one or more cylindrical roll support shafts extending perpendicularly outward from the upright member to receive the tubular core of the roll of non-woven fabric strip.

The filter elements described above do not possess the proper surface properties to efficiently separate water from a hydrocarbon liquid such as fuel. U.S. Publication 2010/0050871 discloses a coalescing media including a polymeric base material having a surface with “air jackets” formed from surface “asperities.” Droplets of the dispersed liquid phase are captured where a layer of air is trapped at the heterogeneous surface and tips of the asperities.

### BRIEF SUMMARY OF THE INVENTION

The performance of a filter media is dependent on its wettability. Hydrophobicity or hydrophilicity of a surface is dependent on the surface energy of the material. For a filter medium, hydrophobicity and hydrophilicity also depends on porosity and pore size, which can also be related to capillary pressure. Materials with lower surface energy yield a hydrophobic surface i.e., water contact angle of 90 degrees and above. However, even a material with a very low surface energy gives a water contact angle of only around 120°. One of the ways to improve the hydrophobicity is to increase the surface roughness in order to reduce the area of contact between the surface and liquid. To achieve higher hydrophobicity, a low surface energy surface with an appropriate surface roughness is required. Therefore, as defined herein, surface energy is a combined effect of hydrophobicity and surface roughness.

To date, there have been no reports on the fabrication of filter elements with very high hydrophobic or super-hydrophobic surfaces via coating methods that can simultaneously provide a solid surface with appropriate surface roughness and low surface energy.

Accordingly, the instant invention provides a filter element with a rough and modified surface for removing a dispersed liquid phase from a continuous liquid phase.

In one aspect, a hydrophobic non-woven, and preferably a synthetic non-woven, filter element with an appropriate surface roughness and therefore low surface energy is provided. A hydrophobic non-woven media made from nanofibers has smaller pores, giving it better water repelling ability than a hydrophobic woven media. The smaller the fibers, e.g. nanofibers, the higher the surface roughness. In turn, the higher surface roughness provides a lower surface energy and therefore affords a higher contact angle than, for example, a media with larger micronic fibers.

The low surface energy filter element described herein is designed to have improved removal of a dispersed liquid phase, such as water, from a continuous liquid phase, such as a hydrocarbon. The separation efficiency of this filter is higher than prior art filters due to its rough surface and low surface energy.

In one embodiment, the low surface energy filter element is prepared by modifying a hydrophobic surface of fine fibers, such as nanofibers or microfibers. The surface is modified by any available surface energy modification techniques that possess the required low surface energy, such as, for example, dip coating, vapor based coating or plasma coating. The treated nanofibers have very fine surface irregularities, making the media more hydrophobic and thereby lowering the surface energy, wherein the contact angles of water droplets at the surface of the filter element exceed 120°.

A method of filtering using the low surface energy filter element as described above or according to any of the embodiments herein comprises arranging the low surface energy filter element in a continuous phase liquid comprising a hydrocarbon liquid stream; and separating a dispersed liquid phase comprising water from the hydrocarbon liquid stream with the low surface energy filter element. As an example, the hydrocarbon liquid stream (continuous phase) is a fuel.

The low surface energy filter element can also be used in gas filter, i.e. such as a natural gas filter.

Other aspects, objectives and advantages of the invention will become more apparent from the following detailed description when taken in conjunction with the accompanying drawings.

### BRIEF DESCRIPTION OF THE DRAWINGS

The accompanying drawings incorporated in and forming a part of the specification illustrate several aspects of the present invention and, together with the description, serve to explain the principles of the invention. In the drawings:

FIG. 1 is a Cassie-Baxter model depicting a micron-sized water droplet captured on a low surface energy non-woven separator.

FIG. 2 is an image of water droplet on the surface of a P100 TW treated media according to an embodiment of the invention.

FIG. 3 is an image of water droplet on the surface of a P 1000 TW treated media according to an embodiment of the invention.

FIG. 4 is an image of water droplet on the surface of Prior Art #1 treated media according to an embodiment of the invention.

FIG. 5 is an image of water droplet on the surface of Prior Art #2 treated media according to an embodiment of the invention.

FIG. 6 is a perspective view in partial section of a multi-overlapped coreless filter media that can be used in any of the embodiments of the invention.

FIG. 7 is a cross-sectional view that illustrates the multi-overlapped coreless filter media of FIG. 6 being formed on a hollow mandrel.

FIG. 8 is a side view of a multi-overlapped coreless filter media circumscribed by an annular seal holder.

FIG. 9 is an enlarged view of the chevron-type seal and seal holder of the filter media of FIG. 8 taken at III.

FIG. 10 is a partial cross-sectional view of the chevron-type seal and the seal holder of FIGS. 8 and 9.

FIG. 11A illustrates a cross-sectional view of a multi-overlapped coreless filter media having an interlaying band in accordance with another embodiment of the present invention; FIG. 11B illustrates a strip for forming an interlaying band positioned against a surface of a strip for forming a band of the filter element for simultaneous winding to provide the configuration shown in FIG. 11A.

FIG. 12 illustrates a cross-section view of another multi-overlapped coreless filter element having an interleafing band in accordance with one embodiment of the present invention.

FIG. 13 illustrates a cross-section view of another multi-overlapped coreless filter element having an interleafing band in accordance with one embodiment of the present invention.

While the invention will be described in connection with certain preferred embodiments, there is no intent to limit it to those embodiments. On the contrary, the intent is to cover all alternatives, modifications and equivalents as included within the spirit and scope of the invention as defined by the appended claims.

### DETAILED DESCRIPTION OF THE INVENTION

As will be appreciated, a hydrophobic rough-surfaced and thereby low surface energy non-woven filter element is disclosed. Preferably, the non-woven media is synthetic. Exemplary filtration applications using various embodiments of a rough-surfaced, low surface energy hydrophobic non-woven filter element are described below with reference to the drawings.

The hydrophobicity of a material, or its tendency to repel water, may be determined by the contact angle of a water droplet on the surface. In general, hydrophobicity is achieved by lowering the surface energy. Thus, non-hydrophobic materials may be rendered hydrophobic by applying a surface coating of low surface energy material. Chemically this may be done, for example, by incorporating apolar moieties, such as methyl or trifluoromethyl groups, into the surface. This results in a material wherein the water contact angle is only around 120° or less.

Thus, in accordance with embodiments of the invention, and to overcome the deficiencies of the prior art, a filter element that can simultaneously provide a surface with appropriate surface roughness and low surface energy is provided. On a rough and hydrophobic surface, the continuous phase fluid, for example, air, natural gas or a hydrocarbon liquid, can be trapped underneath the water droplet which greatly reduces the actual liquid/solid contact area and thus the contact angle increases. The low surface energy filter element according to embodiments has improved removal of dispersed water from a continuous liquid phase, such as, for example, a hydrocarbon, including various types of fuels.

In a preferred embodiment, the non-woven filter elements of the invention exhibit properties approaching or even reaching “superhydrophobic.” As defined herein, “superhydrophobic” properties refer to having water contact angles larger than about 150° and theoretically up to 180°. Superhydrophobic filter media has self-cleaning behavior and hence has a longer life. As such, the filter element of the invention has a surface wherein a dispersed liquid, preferably water, has a contact angle at the surface of the filter element exceeding 120°, preferably exceeding 130°, and more preferably exceeding 140° or even 150° and 160°. By “contact angle” is meant the angle (measured through a continuous liquid unless stated otherwise) at which a liquid interface meets a solid surface.

Before proceeding further, it may be useful to define some of the other terms being used herein. “Pore size” is an indication of the size of the pores in the media, which determines the size of particles unable to pass through the media, i.e. micron rating. For most media, this may be related as a distribution, since the pore size may not be uniform throughout. Average pore size can be determined by various methods known to those skilled in the art, such as, for example, manually. Typically, some of the embodiments discussed herein will have an average pore size of between 30 and 180 micron, with a minimum pore size of about 15 micron. Nanofibers can reduce effective pore size to between 0.50 and 1.00 micron (with a minimum pore size of about 0.25 micron and a maximum pore size of about 1.50 micron), such that average pore size can be measured prior to deposition of nanofiber. “Fiber size” is a measure of the size of the fibers in the media. This is measured in microns, denier, or preferably according to the instant invention, nanometers (nm). Generally, the smaller the fiber, the smaller the pores in the media. There is generally a distribution of fiber sizes which can change based upon design. “Basis Weight” is how much the media weighs for a given surface area. This is generally measured in pounds (lbs.) per square yard, or grams per square meter. “Porosity” (Void volume) is a measure of how much of the media volume is open space. Generally, a higher porosity indicates a higher dirt holding capability within the media and a higher permeability. Fuzziness can be determined by surface roughness and/or by the provision of free terminating ends of fibers. In some embodiments, terminating ends of fibers will be freely projecting generally in a cantilever manner from the upstream surface of the media, which when stretched straight measure greater than 3 millimeters. More than one of these freely projecting fibers may be contained in a square centimeter of media surface on average.

Oleophilic properties, i.e. having a strong affinity for oily substances, can be measured using Isopar™ contact angles. Isopar™ fluids are high-purity synthetic isoparaffins (branched-chain alkanes) with consistent and uniform quality. As defined herein, when a droplet of Isopar™ on the surface of a filter element of the invention has a contact angle of less than 90°, the filter media is considered to be oleophilic in nature. Conversely, when a droplet of Isopar™ on the surface of a filter element of the invention has a contact angle of more than 90°, the filter media is considered to be oleophobic in nature.

In a preferred embodiment, filter media that are modified according to the present invention are those described in U.S. Pat. Nos. 5,827,430; 5,893,956; 5,919,284; 6,168,647 and 8,062,523, all incorporated herein by reference, and marketed by the Perry Equipment Corporation of Mineral Wells, Tex. (PEACH®). For example, the PEACH® filter media disclosed in U.S. Pat. Nos. 5,827,430 and 5,893,956 consists of multiple layered sections of media formed into a conical helix pattern. The media can be made of at least one non-woven fabric of a homogeneous mixture of a base and a binder material that is compressed to form a mat or sheet of selected porosity. The binder fiber has at least a surface with a melting temperature lower than that of the base fiber. The sheet is formed into a selected geometric shape and heated to thermally-fused to bind the base fiber into a porous filter element. The preferred shape is a helically wound tube of plural sheets, each sheet being self-overlapped and compressed to overlap another sheet. Each sheet is preferably heated and compressed individually and the sheets may be selected to have different porosities and densities. The binder material is selected from the group consisting of thermoplastic and resin, and the base material is selected from the group consisting of thermoplastic and natural. A plurality of these filter media can be used. Each media can also include at least one band of base media having a selected porosity and an interlay having a different porosity within at least one band of the base media. Regardless, each filter media usually employs one or more, and preferably at least two to four, multi-overlapped non-woven strips, wherein each strip is wrapped multiple times upon itself, and wherein each strip is made of a different type of fiber. Alternatively, the filter is not formed into a conical helix pattern but is sheet material that is optionally pleated or formed as a cylindrical sleeve and mounted to a support core.

Each non-woven fabric strip is composed of selected polymeric fibers such as polyester and polypropylene which serve as both base fibers and binder fibers. Base fibers have higher melting points than binder fibers. The role of base fibers is to produce small pore structures in the coreless filter element. The role of the binder fiber or binder material is to bond the base fibers into a rigid filter element that does not require a separate core. The binder fibers may consist of a pure fiber or of one having a lower melting point outer shell and a higher melting point inner core. If the binder fiber is of the pure type, then it will liquefy throughout in the presence of sufficient heat. If the binder fiber has an outer shell and an inner core, then it is subjected to temperatures that liquefy only the outer shell in the presence of heat, leaving the inner core to assist the base fiber in producing small pore structures. The role therefor of the binder fiber is to liquefy either in whole or in part in the presence of heat, the liquid fraction thereof to wick onto the base fibers to form a bond point between the base fibers, thereby bonding the base fibers together upon cooling. The binder material may be in a form other than fibrous.

In accordance with the invention, many techniques are employed in the present invention to render surfaces hydrophobic, or provide existing hydrophobic surfaces with an even lower surface energy. Examples include dip-coating, plasma polymerization or etching of apolar polymers like polypropylene, polytetrafluoroethylene, chemical vapor deposition, sublimation material and paint or sprays containing hydrophobized microbeads or evaporation of volatile compounds, and the like. Preferably, a plasma coating method is employed as described, for example, in U.S. Pat. No. 6,419,871, which is incorporated herein by reference. Specifically, a media is treated with a fluorine-containing plasma to create a deposition of about 0.03 g/m² to about 1.5 g/m² of a fluoropolymer.

The plasma treatment as disclosed in the ‘871 patent uses a fluorine-containing plasma. This means that the plasma contains a fluorine source such that a fluorine free radical or ion is formed. The fluorine source can be elemental fluorine or a fluorine-containing compound. Examples of suitable fluorine sources include short chain fluorocarbons having 1 to 8 carbon atoms, preferably 1-3 carbon atoms, wherein at least one hydrogen atom has been replaced with a fluorine atom. Preferably, at least 25 mol % of the hydrogen atoms have been replaced with fluorine atoms, more preferably at least 50%. The fluorocarbons can be saturated or unsaturated. Other fluorine sources include fluorosilanes. Concrete examples of fluorine sources include fluorine, trifluoromethane, tetrafluoroethane, and tetrafluorosilane (SiF₄).

The plasma is typically comprised of the fluorine source, only, although other materials can be present. In one embodiment, the fluorine source is mixed with a carrier gas such as nitrogen, which may cause higher fluorine radical generation in the plasma.

Suitable plasma conditions to ensure deposition of about 0.03 g/m² to about 1.5 g/m², preferably about 0.05 g/m² to 1.0 g/m², more preferably about 0.07 g/m² of a fluoropolymer can be readily determined by conventional means. The power, duration, and pressure can vary significantly depending on the size and shape of the chamber and the composition of the plasma. In general, the power ranges from 10 to 5000 watts, the duration of the treatment is from one second to five minutes and the process pressure is from 10 milliTorr to 1000 milliTorr. Subsequent to plasma treatment the filter is washed in an aqueous solvent mixture, such as an isopropyl alcohol/water mixture, or water, and dried.

In a particular, non-limiting embodiment of the invention, a low surface energy filter element is made by modifying at least one surface of a high surface energy media of fine fibers, such as, for example, nanofibers. Nanofibers are fine fibers formed from electrospinning or electrostatic melt blowing with average diameter (e.g. thickness) less than one micron and typically less than 800 nanometers, preferably less than 500 and in some embodiments less than 200 nanometers. The fine fibers can either be on the surface of a substrate layer or integrated into a media layer. For example, it is contemplated that one way to improve the efficiency, reduce pore size (without necessarily increasing restriction) and capabilities of filter media includes the use of extremely fine fibers, or nanofibers, such as disclosed in application Ser. No. 12/271,322, entitled Filtration medias, Fine Fibers Under 100 nanometers and methods; application Ser. No. 12/428,232, entitled Integrated Nanofiber media; application Ser. No. 12/357,499 entitled Filter Having Meltblown and Electrospun fibers, the entire disclosures of which are hereby incorporated by reference. Such embodiments and broader claimed aspects relate to contemplated use of such nano-fibers to provide for tiny pores for mist filtration. These fine fibers may be made from a variety of different polymers (thermoplastic and natural) as generally disclosed in the aforesaid publications, such as, for example, nylon, a polyvinylidene fluoride (PVDF), a polyurethane (PU), a polyacrylonitrile (PAN), a cellulose Tri Acetate (CTA), a polymethylmethacrylate (PMMA), a poly(vinylidene fluoride-co-hexafluoropropene) (PVDF-HFP), a poly (4-methyl-1-pentene) (PFMOP) and a polytetrafluoroethylene (PTFE). In a more preferred embodiment, at least one low surface energy PEACH® separator is made by using a hydrophobic surface with fine fibers, preferably nanofibers. The high surface energy nanofibers are coated with fluoropolymer, such as, for example, with a plasma coating technique to convert the high surface energy nylon nanofiber media into a low surface energy filter media. The nanofiber surface is modified by any available surface energy modification techniques that provide the required low surface energy, as discussed above.

In a preferred embodiment, two PEACH® stations are used, and 4″ or 6″ width plasma coated nanofiber media are fed on both stations and a helical wound tube is created. The temperatures are adjusted to provide enough thermal bonding and structural strength to the helical tube. It is also possible to use only one station to prepare a thin PEACH® tube. The nanomatrix media is first laminated with P1000/scrim to protect the nanofibers and then plasma coated as described above. Fluoropolymer coating converts the high surface energy nanofiber media into a low surface energy filter element. The resulting filter element utilizes both a rough surface and a low surface energy to provide increased hydrophobicity. The surface energy of filter media according to this embodiment is given in Table 1. Referring to Table 1, it is noted that P100 and P200 have nanofibers electrospun on a polyester substrate. The difference between P100 and P200 media is that they have different amount of nanofibers in them, i.e. P200 has a less amount of nanofibers compared to P100. It is noted that P1000 is a media that contains no nanofiber. As discussed above, the higher the amount of nanofibers, the rougher the filter media surface is, as the media forms many small pores. The hydrophobic media with higher surface roughness in turn has higher water repelling ability than the hydrophobic media with low surface roughness.

TABLE 1

| | | | | | Contact angle (degrees) | |
|---|---|---|---|---|---|---|
| | | Pore size (micron) | | | | Isopar ™ |
| Filter media | Fiber size | Average | Minimum | Maximum | Water (θ_{w}) | (θ_{Oil}) |
| P100 | 75-150 | 0.57 | 0.27 | 1.06 | 141.9 ± 1.26 | ~0 |
| LC TW | nm | | | | | |
| P200 | 75-150 | 0.70 | 0.31 | 1.45 | 136.9 ± 3.25 | ~0 |
| LC TW | nm | | | | | |
| P1000 | 17 micron | 50.20 | 16.27 | 110.40 | 133.7 ± 1.06 | 72.3 ± 11.34 |
| TW | | | | | | |

TW: Fluorocarbon coated media with plasma coating technique as described herein

In contrast, the existing industry standard separator media is in woven form and made with fibers around 37-110 micron size fibers. To this end, the surface energy of existing filter media in terms of water and Isopar contact angle is given in Table 2.

TABLE 2

| | | Average fiber | Average pore | Contact angle (degrees) | |
|---|---|---|---|---|---|
| Filter media | Micron rating | size (μm) | size (μm) | Water | Isopar ™ |
| Woven coated | 25 μm screen | 37.12 | 25 | 132.0 ± 1.76 | ~0 |
| synthetic media - | SM | | | | |
| Prior Art #1 | | | | | |
| Woven Teflon | micron rating of | 111.81 | 75 | 134.1 ± 2.03 | 80.1 ± 3.40 |
| separator media - | 74 μm | | | | |
| Prior Art #3 | | | | | |
| Woven coated | 52 μm Separator | 41.03 | 50 | 140.3 ± 1.58 | 116.2 ± 3.62 |
| synthetic media - | Synthetic screen | | | | |
| Prior Art #2 | | | | | |

Accordingly, and referring to FIG. 1, the filter element of the invention is therefore very useful for liquid-liquid separation, since most of the droplets of a dispersed liquid phase are micron sized, and are trapped by the surface irregularities of a surface modified nanofiber-sized filter element that is immersed in a continuous liquid phase. Since the surface of the filter element is hydrophobic, a dispersed water droplet 1 cannot penetrate into the grooves or pockets 3 created by the surface irregularities 5 of the nanofibers. As depicted in FIG. 1, this is known as a Cassie-Baxter state, wherein the water droplet 1 is resting on the tops of the irregularities 5 (as opposed to being in intimate contact with the same, as in the Wenzel state), or in other words, on top of a composite media surface consisting of a continuous hydrocarbon liquid and the filter media. As a nanofiber filter element of the invention is immersed in a hydrocarbon liquid, the spaces between the irregularities fill with hydrocarbon 7, leaving the dispersed water droplet 1 to rest on the composite media of hydrocarbon and filter media. Water angle is measured by methods known by those skilled in the art, including but not limited to, the static sessile drop method (via a goniometer), the dynamic sessile drop method, and the like.

In sharp contrast, a treated filter element made with micron or denier size fibers generates bigger pores as compared to the nanofiber filter media of the invention. In addition, this larger sized media is inadequate to generate fine surface irregularities as its nanofiber filter element counterpart. Hence, a micron sized dispersed water droplet cannot rest on a composite media of hydrocarbon and filter media. Since a hydrocarbon and water repel each other, a PEACH® filter element prepared with hydrophobic nanofibers has a higher water separation efficiency under a Cassie-Baxter state (FIG. 1) than does a filter element made with micron or denier sized fibers. As a result, a filter media made of non-woven, submicron fibers is preferred for modification according to many or certain embodiments of the invention.

In another embodiment, a PEACH® filter element is prepared with two different hydrophobic non-woven media, including but not limited to, a fluorocarbon coated Perry

Engineered media (PEM) (PECOFacet Engineered media); a fluoropolymer non-woven media, preferably ethylene chlorotrifluoroethylene (ECTFE), polyvinylidene fluoride (PVDF), poly(vinylidene fluoride-co-hexafluoropropene) (PVDF-HFP) or polytetrafluoroethylene (PTFE); or another hydrophobic polymer (thermoplastic and natural) such as, for example, a polyurethane (PU), a polyacrylonitrile (PAN), a cellulose tri-acetate (CTA), or a polymethylmethacrylate (PMMA), a polystyrene or a plasma coated nanofiber. In a more preferred embodiment, polyethylene terephthalate (PET) PEM is used as one of the hydrophobic non-woven media. In a preferred embodiment, the filter element is designed in such a way that the outside of the PEACH® tube has a ECTFE or PVDF media, with a PEM/fluorocarbon coated PEM remaining closer to the core. Machine temperatures are adjusted to bond the PEM and the fluoropolymer non-woven media to each individually as well as to each other. Again, two stations are utilized, with low melt PEM media fed on a first station, which is closer to the core, and the low surface energy/high melt fluoropolymer media used on a second station so that it overlaps on low melting high surface energy media (PEM) and both are bonded together. Both the media are wrapped in such a way that the media (PEM) always remains closer to the core and never actually reaches the outside surface of the tube. This ensures improved surface roughness, which corresponds to lower surface energy.

According to this particular embodiment, fluorocarbon coated PEM is hydrophobic and fuzzy, with fibers standing out from the surface of the media. Fluorpolymer non-woven media such as ECTFE is also hydrophobic and rough in nature. Thus, a filter element prepared from PEM and ECTFE (or PVDF) media, or PEM and nanomatrix media, provides a duel roughness created by the surface roughness of the media itself and the fuzzy hydrophobic fibers. This can be compared to the famous Lotus leaf effect wherein the superhydrophobicity of the lotus leaf is result of a “hierarchical double structure” formed out of a rough-surfaced epidermis (in the form of papillae) and the covering waxes imposed thereon. Duel roughness of the filter media results in a composite surface further enhancing hydrophobicity of the media. Improved hydrophobicity of the filter media enhances its water separation performance based on the Cassie-Baxter model as shown in FIG. 1. It is noted that PEM has lower melting point as compared to the ECTFE or PVDF polymer. The surface energy of media prepared by this embodiment of the method is listed in Table 3.

TABLE 3

| | | | | | Contact angle (degrees) | |
|---|---|---|---|---|---|---|
| | | Pore size (micron) | | | Water | Isopar ™ |
| Filter media | Fiber size | Average | Minimum | Maximum | (θ_{w}) | (θ_{Oil}) |
| Halar ® ECTFE | 24.42 | 140.75 | 59.27 | 260.47 | 131.8 ± 1.70 | ~0 |
| spunbond | micron | | | | | |
| (2.8 opsy) | | | | | | |
| Halar ® ECTFE | 23.45 | 149.05 | 65.42 | 258.76 | 131.8 ± 1.70 | ~0 |
| spunbond | micron | | | | | |
| (3 opsy) | | | | | | |
| Halar ® ECTFE | 23.37 | 112.44 | 48.78 | 233.61 | 131.8 ± 1.70 | ~0 |
| spunbond | micron | | | | | |
| (6 opsy) | | | | | | |
| PET PEM | 12DBC/60D | 146.22 | 28.53 | 445.76 | 126.3 ± 3.63 | ~0 |
| #1* | | | | | | |
| PET PEM | 12DBC/90D | 172.81 | 54.77 | 427.5 | 125.2 ± 2.69 | ~0 |
| #2* | | | | | | |
| PET PEM | 3DBC/1.4D | 151.74 | 43.88 | 382.89 | 135.4 ± 1.46 | ~0 |
| #3* | | | | | | |
| PET PEM | 3DBC/6D/ | 128.20 | 53.18 | 258.74 | 135.0 ± 2.11 | ~0 |
| #4* | 15D | | | | | |

DBC: denier bicomponent fiber
D: denier
*Fluorocarbon coated media with plasma coating technique as described herein

In yet another embodiment, a PEACH® filter element is prepared with polymers (thermoplastic and natural) such as low surface energy ECTFE or plasma coated nanomatrix interlay. In this embodiment, nanomatrix or ECTFE media is interlaid on top of PEM media (see U.S. Pat. Nos. 8,062,523 and 8,293,106, incorporated herein by reference). Two stations are employed, wherein ECTFE or plasma coated nanomatrix media is fed on a first station, second station or both stations. Media prepared by this embodiment of the method is provided for in Table 4. The hydrophobic PEM has fuzzy fibers, which along with the surface roughness of the media, are bonded to the fine rough nanomatrix surface creating a 3-D matrix which has improved surface irregularities. This media has higher water repelling ability due to its surface roughness.

It is noted from the water contact angle information provided in Table 4 below for various filter elements of the invention, PET PEM #5 and PET PEM #6 media, when heat laminated, reduce the fuzziness of such media, and hence the water contact angle on the media decreases (PET PEM #5 and PET PEM #6 are also not plasma treated). This shows that the fuzziness of the filter media helps in lowering the surface energy of the media by creating a “rough” surface laminated on the hydrophobic nanofiber surface and creating duel roughness by further lowering the surface energy of the media. As used herein, low surface energy of the filter element corresponds to higher water separation efficiency.

TABLE 4

| | | | | | Contact angle (degrees) | |
|---|---|---|---|---|---|---|
| Filter | | Pore size (micron) | | | | Isopar ™ |
| media | Fiber size | Average | Minimum | Maximum | Water (θ_{w}) | (θ_{Oil}) |
| PET | 12DBC/90D/150D | 65.09 | 5.95 | 25344.45 | 113.1 ± 1.21 | ~0 |
| PEM | | | | | 109.2 ± 3.65 | |
| #5 | | | | | (After lamination) | |
| PET | 3DBC/6D/15D | 128.20 | 53.18 | 258.74 | 124.7 ± 1.99 | ~0 |
| PEM | | | | | 122.1 ± 0.5 | |
| #6 | | | | | (After lamination) | |

DBC: denier bicomponent fiber;
D: denier

Surface energy measurements are also performed by placing a static water droplet on a filter media of the invention after the filter media is immersed in a Kerosene-type fuel, such as for example, Jet-A without additives. The results are given in Table 5 below. Where the water contact angles on filter media is greater than 150°, the media is considered as superhydrophobic, has “self cleaning” behavior and is therefore deemed to have a longer life. All of the media described in table 3 are hydrophobic (and most are superhydrophobic) and repel water. FIG. 2 is an image of water droplet on the surface of a P100 TW treated media for which the water contact angle is calculated. FIG. 3 is an image of water droplet on the surface of a P1000 TW treated media for which the water contact angle was calculated. FIG. 4 is an image of water droplet on the surface of a #2 treated media for which the water contact angle is calculated. FIG. 5 is an image of water droplet on the surface of a #1 treated media for which the water contact angle is calculated.

TABLE 5

| | | Water contact | |
|---|---|---|---|
| Filter media | Structure | angle (θ_{W}) | Observation |
| Prior | Woven | 162.8 ± 8.53 | Water drop rolls on the |
| Art #2 | | | surface, self-cleaning |
| | | | behavior (see FIG. 4) |
| Prior | Woven | 141.1 ± 8.00 | Strongly repels water |
| Art #1 | | | drops (see FIG. 5) |
| Prior | Woven | 158.8 ± 15.47 | Water drop rolls on the |
| Art #3 | | | surface, self-cleaning |
| | | | behavior |
| P100 TW | Non-woven | 163.7 ± 7.43 | Strongly repels water |
| | | | drops (see FIG. 2) |
| P1000 TW | Non-woven | 142.4 ± 4.88 | Strongly repels water |
| | | | drops (see FIG. 3) |
| PET PEM | Non-woven | 143.4 ± 6.36 | Fuzzy fibers with |
| #3 | | | hydrophobic coating |
| | | | makes the drops roll on |
| | | | the media, self-cleaning |
| | | | property of the media |
| Halar ® | Non-woven | 150.0 ± 2.40 | Water drop rolls on the |
| (ECTFE) 6 | | | surface, self-cleaning |
| OZSY basis | | | behavior |
| weight | | | |

It is noted that since water and oil repel each other, the water contact angles reported in Table 5 are higher than any of those reported in Tables 1-4. Table 5 clearly shows that when the media of the invention is immersed in Jet-A fuel, it retains its hydrophobicity.

Testing of filters and separators was performed according to the provisions of “Specifications and Qualification Procedures for Aviation Jet Fuel/Separators”, API/IP Specification 1581, 5th Edition, July 2002. Generally, to verify the procedure, prior art filters are tested with separator Prior Art #1 (with coalescer) or Prior Art #2 (no coalescer). All separators according to the instant invention are then tested (with or without coalescer TC-00162). API/IP Specification 1581 requires the separators to be tested for water removal as well as solids loading ability. For the solids loading testing described here the separators are tested without a coalescer. The water removal efficiency testing is carried out with the presence of a coalescer. The flow rate through the separator, using Category C fuel (commercial aviation fuel), is 30 gpm (U.S. gallons per minute) on recirculation basis, with 0.5% water and then 3.0% water for 30 minutes. Water content samples are read at 5, 10, 20 and 30 minutes (vessel differential pressure, d.p. which is the total pressure accounting the pressure drop across both coalescer and separator and the vessel restriction, measured at each reading). In addition, the pressure drop d.p. across the separator alone was measured and reported. If these tests are successful, the flow rate is increased to 40 gpm with 0.5% and then 3.0% water, respectively, for 30 minutes. If a separator is tested successfully after 40 gpm, testing is repeated with Category M fuel (military aviation fuel).

Tables 6 and 7 represent fuel testing results for prior art filters Prior Art #1 and Prior Art #2 respectively. Prior Art #1 is tested with a military grade EI/IP 1581 5^{th} Edition qualified coalescer. Separators are incapable of handling an emulsion. Hence, the water removal/separation efficiency of the separators is tested in the presence of the coalescer. The coalescer converts the emulsion to droplets, and high water removal efficiency is achieved by using a coalescer and separator together. Prior Art #2 is tested for solids loading ability as Prior Art #2 has larger pores compared to the Prior Art #1. It should be noted that the separators should be able to efficiently separate the water droplets without being loaded with the solids. The solids build up in the separator resulting in increased pressure drop and limit the life of the separator. PET PEM #7 is made with 6 inch wide TW (plasma treated, wherein PET PEM #5 is the same, only not plasma treated) PET PEM media used on station 1 and P100/P1000 TW media used on station 3 of a PEACH® machine. PET PEM #7 media is made up of 12DBC/90D/150D size PET fibers in 50:25:50 proportion and is plasma coated. The 12 DBC is a bi-component staple fiber made up with polybutylene terephthalate (PBT) and PET. PET PEM #8 is made with 6 inch wide plasma treated PET PEM media on station 1 and 3 of a PEACH® machine. PET PEM #9 is made with 6 inch wide plasma treated PET PEM media on station 1 and 2 of a PEACH® machine. The solids load and solids concentration is the amount of solids added to the hydrocarbon liquid upstream of the separator and the amount of solids measured gravimetrically on the downstream of the separator, respectively.

TABLE 6
Separator: Prior Art #1; Coalescer: Military Grade; Fuel Category C

| | | Fuel | ΔP | | | | |
|---|---|---|---|---|---|---|---|
| | | Flow | (psi) | | | Water | |
| | Time | Rate | Separator | ΔP | Water | Conc. | Temp. |
| | (min) | (gpm) | Only | (psi) | Flow Rate | (ppm) | 8 F. |
| Separator | | 30 | 0.5 | | | | 71 |
| D.P. | | 40 | 0.6 | | | | 71 |
| Water .5% | 0 | 30 | 0.5 | 4.5 | .15 gal. | — | |
| | 5 | 30 | 0.5 | | .15 | 0.0 | |
| | 10 | 30 | 0.5 | 9.5 | .15 | 0.0 | |
| | 15 | 30 | 0.5 | 9.9 | .15 | 0.0 | |
| Water 3% | 0 | 30 | 0.5 | 10.3 | .9 gal. | — | |
| | 5 | 30 | 0.4 | 13.3 | .9 | 0.0 | |
| | 10 | 30 | 0.4 | 14.3 | .9 | 0.0 | |
| | 15 | 30 | 0.4 | 15.5 | .9 | 0.0 | |
| Water .5% | 0 | 40 | 0.5 | 11.5 | .2 gal. | 0.0 | |
| | 5 | 40 | 0.5 | 15.8 | .2 | 0.0 | |
| | 10 | 40 | 0.5 | 16.7 | .2 | 0.0 | |
| | 15 | 40 | 0.5 | 16.9 | .2 | 0.0 | |
| Water 3% | 0 | 40 | — | — | 1.2 gal. | — | |
| | 5 | 40 | 0.5 | 21.6 | 1.2 | 0.0 | |
| | 10 | 40 | 0.5 | 24.3 | 1.2 | 0.0 | |
| | 15 | 40 | 0.5 | 26.1 | 1.2 | 0.0 | |
| Water .5% | 0 | 45 | 0.6 | 20.9 | .23 gal. | — | |
| | 5 | 45 | 0.6 | 22.0 | .23 | 0.0 | |
| | 10 | 45 | 0.6 | 21.9 | .23 | 0.0 | |
| | 15 | 45 | 0.6 | 22.5 | .23 | 0.0 | |
| Water 3% | 0 | 45 | 0.6 | 31.0 | 1.35 gal. | — | |
| | 5 | 45 | 0.6 | 31.7 | 1.35 | 0.0 | |
| | 10 | 45 | 0.6 | 34.3 | 1.35 | 0.0 | |
| | 15 | 45 | 0.7 | 37.4 | 1.35 | 0.0 | |

TABLE 7
Separator: Prior Art #2; Coalescer: None; Fuel Category: Non-Additives

| | | Fuel | | | | | | |
|---|---|---|---|---|---|---|---|---|
| | | Flow | | | Solids | Solids | | |
| | Time | Rate | ΔP | | Rate | Conc. | Sample | Temp. |
| Phase | (min) | (gpm) | (psi) | K (pS/m) | [symbol] mg/l | (mg/l) | Size | [symbol] ° F. |
| Solids Holding | 0 | 35 | 0.3 | 000 | 5 mg/l | | | 70 |
| Test | 5 | 35 | 0.3 | | 5 | 3.05 | 5 L | 70 |
| | 10 | 35 | 0.3 | | 5 | | | 70 |
| | 20 | 35 | 0.3 | | 5 | 3.00 | 5 L | 70 |
| | 30 | 35 | 0.3 | | 5 | | | 70 |
| | 40 | 35 | 0.3 | | 5 | 3.20 | 5 L | 70 |
| | 50 | 35 | 0.3 | | 5 | | | 70 |
| | 60 | 35 | 0.3 | | 5 | 3.26 | 5 L | 70 |

Tables 8-12 represent fuel testing results for filters of the invention. Table 13 represents fuel testing for Prior Art #1.

TABLE 8
Separator: PET PEM #8; Coalescer: Military Grade Coalescer;
Fuel Category C

| | | Fuel | ΔP | | | |
|---|---|---|---|---|---|---|
| | | Flow | (psi) | | Water | Water |
| | Time | Rate | Separator | ΔP | Flow | Conc. |
| | (min) | (gpm) | Only | (psi) | Rate | (ppm) |
| Separator | | 30 | 0.3 | 4.7 | | |
| D.P. | | 40 | 0.4 | 6.2 | | 0.0 |
| Water .5% | 0 | 30 | 0.3 | 4.8 | .15 gal. | — |
| | 5 | 30 | 0.2 | 8.9 | .15 | 0.0 |
| | 10 | 30 | 0.2 | 9.5 | .15 | 0.0 |
| | 15 | 30 | 0.3 | 9.5 | .15 | 0.0 |
| Water 3% | 0 | 30 | 0.3 | 9.7 | .9 gal. | — |
| | 5 | 30 | 0.2 | 12.4 | .9 | 0.0 |
| | 10 | 30 | 0.3 | 12.7 | .9 | 0.0 |
| | 15 | 30 | 0.3 | 13.3 | .9 | 0.0 |
| Water .5% | 0 | 40 | 0.3 | 11.9 | .2 gal. | — |
| | 5 | 40 | 0.3 | 14.8 | .2 | 0.0 |
| | 10 | 40 | 0.3 | 15.4 | .2 | 0.0 |
| | 15 | 40 | 0.3 | 15.6 | .2 | 0.0 |
| Water 3% | 0 | 40 | 0.3 | 15.8 | 1.2 gal. | — |
| | 5 | 40 | 0.3 | 18.8 | 1.2 | 0.0 |
| | 10 | 40 | 0.3 | 19.8 | 1.2 | 0.0 |
| | 15 | 40 | 0.3 | 20.6 | 1.2 | 0.0 |
| Water .5% | 0 | 45 | 0.4 | 15.4 | .23 gal. | — |
| | 5 | 45 | 0.4 | 16.6 | .23 | 0.0 |
| | 10 | 45 | 0.3 | 20.1 | .23 | 0.0 |
| | 15 | 45 | 0.3 | 20.6 | .23 | 0.0 |
| Water 3% | 0 | 45 | 0.4 | 20.8 | 1.35 gal. | — |
| | 5 | 45 | 0.3 | 24.9 | 1.35 | 0.2 |
| | 10 | 45 | 0.3 | 26.1 | 1.35 | 0.5 |
| | 15 | 45 | 0.4 | 26.9 | 1.35 | 2.1 |

TABLE 9
Separator: PET PEM #8; Coalescer: None;
Fuel Category: Non-Additives

| | | Fuel | | | | | | |
|---|---|---|---|---|---|---|---|---|
| | | Flow | | | Solids | Solids | | |
| | Time | Rate | ΔP | K | Rate | Conc. | Sample | Temp. |
| Phase | (min) | (gpm) | (psi) | (pS/m) | [symbol] mg/l | (mg/l) | Size | [symbol] ° F. |
| Solids | 0 | 35 | 0.2 | 000 | 5 mg/l | | | 68 |
| Holding | 5 | 35 | 0.2 | | 5 | 2.00 | 5 L | 69 |
| Test | 10 | 35 | 0.2 | | 5 | | | 69 |
| | 20 | 35 | 0.2 | | 5 | 2.78 | 5 L | 69 |
| | 30 | 35 | 0.2 | | 5 | | | 69 |
| | 40 | 35 | 0.2 | | 5 | 3.05 | 5 L | 69 |
| | 50 | 35 | 0.2 | | 5 | | | 69 |
| | 60 | 35 | 0.2 | | 5 | 3.26 | 5 L | 69 |

TABLE 10
Separator element: PET PEM #7; Coalescer: Commercial
Grade Coalescer; Length of Separator: 6 inches; Length
of Coalescer: 14 inches; Interfacial Tension (IFT): 22.80
dyne/cm; Initial DP: 2.8 psid at 16 gpm; Surface Tension
of Isopar ™: 38.03 dyne/cm. Test liquid: Isopar ™

| | | Water | Total | | |
|---|---|---|---|---|---|
| | | Injection | Pressure | Water Cons. | |
| | Flow | Rate | Drop | (ppm) | |
| Time | Rate | (ml/min) | (PSID) | Inlet | Outlet |
| 2.14 PM | 16 GPM | 48 | 3.6 | 316.52 | 0.13 |
| 2.38 PM | 20 GPM | 48 | 5 | 315.87 | 0.055 |
| 2.45 PM | 20 GPM | 48 | 5.3 | 315.87 | 0.055 |
| 2.46 PM | 25 GPM | 48 | 6.5 | 270.97 | 0.103 |
| 3.04 PM | 30 GPM | 48 | 7.9 | 222.58 | 0.097 |
| 3.25 PM | 30 GPM | 80 | 8.1 | 388.46 | 0.33 |
| 3:45 PM | 36 GPM | 80 | 8.7 | 304.85 | 0.47 |

TABLE 11
Separator element: PEACH ® P100/P1000 TW at Stations
1 and 3; Coalescer: Commercial Grade Coalescer; Length of Separator:
6 inches; Length of Coalescer: 14 inches; Interfacial Tension
(IFT): 22.80 dyne/cm; Initial DP: 5.8 psid at 16 gpm; Surface
Tension of Isopar ™: 38.03 dyne/cm. Test liquid: Isopar ™

| | | | Water | Total | Jorin ViPA (Water | |
|---|---|---|---|---|---|---|
| | | | injection | Pressure | concentration | |
| | | Flow | rate | drop | (PPM)) | |
| Time | | rate | (ml/min) | (PSID) | Inlet | Outlet |
| 9:21 | AM | 16 GPM | 48 | | 268 | 0.11 |
| 9:30 | AM | 16 GPM | 48 | | 268 | 0.03 |
| 9:36 | AM | 16 GPM | 48 | 6.6 | 268 | 0.22 |
| 10 | AM | 20 GPM | 48 | 9.3 | 325 | |
| 10:15 | AM | 25 GPM | 48 | 12.4 | 280 | 0.12 |
| 10:30 | AM | 30 GPM | 48 | 15.1 | 250 | 0.39 |
| 11 | AM | 36 GPM | 80 | 18.2 | 304 | 0.22 |

TABLE 12
Separator element: PET PEM #9; Coalescer: Commercial Grade Coalescer; Length of
Separator: 6 inches; Length of Coalescer: 14 inches; Interfacial Tension (IFT): 38.27
dyne/cm; Surface Tension of Isopar ™: 38.03 dyne/cm. Test liquid: Isopar ™

| | | | | | Jorin ViPA | |
|---|---|---|---|---|---|---|
| | | | | | (Water | |
| | | | Total | | concentration | |
| | Flow | Water injection | Pressure drop | Temperature | (PPM)) | |
| Time | rate | rate (ml/min) | (PSID) | (0 F.) | Inlet | Outlet |
| 10:30 | 16 GPM | 48 | 3.2 | 84 | 506 | 0.26 |
| AM | | | | | | |
| 10:56 | 20 GPM | 48 | 5.6 | 85.3 | 430 | 0.49 |
| AM | | | | | | |
| 11:13 | 25 GPM | 48 | 6.9 | 85.5 | 378 | 0.16 |
| AM | | | | | | |
| 11:25 | 30 GPM | 48 | 8.1 | 86.6 | 255 | 0.91 |
| AM | | | | | | |
| 11:50 | 30 GPM | 80 | 8.5 | | 257 | 0.83 |
| AM | | | | | | |
| 12:10 | 36 GPM | 80 | 9.7 | 86.7 | 275 | 0.63 |
| PM | | | | | | |

TABLE 13
Separator element: Prior Art #1; Coalescer: Commercial
Grade Coalescer; Length of Separator: 6 inches; Length
of Coalescer: 14 inches; Interfacial Tension (IFT): 38.27
dyne/cm; Initial DP: 2.0 psid at 16 gpm; Surface Tension
of Isopar ™: 38.03 dyne/cm. Test liquid: Isopar ™

| | | Water | Total Pressure | Jorin ViPA (Water | |
|---|---|---|---|---|---|
| | | injection | drop (PSID) | concentration | |
| | Flow | rate | (coalescer + | (PPM)) | |
| Time | rate | (ml/min) | separator) | Inlet | Outlet |
| 3:25 PM | 16 GPM | 48 | 3.6 | 261.18 | 0.29 |
| 3:40 PM | 20 GPM | 48 | 4.9 | 364.96 | 0.32 |
| 3:30 PM | 25 GPM | 48 | 6.5 | 271.99 | 4.28 |
| 3:44 PM | 30 GPM | 48 | 8 | 272 | 3.72 |
| 3:53 PM | 30 GPM | 80 | 8.3 | 528 | 2.92 |
| 3:59 PM | 36 GPM | 80 | 9.8 | 306.7 | |
| 4:04 PM | 36 GPM | 80 | 10.1 | | 0.22 |

As discussed above, a preferred embodiment of the invention utilizes PEACH® filter media as disclosed in, for example, U.S. Pat. Nos. 5,827,430 and 5,893,956. The total thickness of the filter media can vary, but preferably has considerable depth wherein fluid may pass through a substantial depth of filter media through which particulates may be deposited throughout the depth thereof For example, a typical filter media layer thickness of depth media may be at least ¼ of an inch and preferably at least ½ of an inch. Examples of such depth media, which are commonly sold under the trade designation PEACH, are illustrated and disclosed in U.S. Pat. No. 5,827,430. Specifically, referring to FIG. 6 of the drawings, the numeral 11 designates an example of a multi-overlapped coreless filter media used to provide the filter media of the invention. It includes a first multi-overlapped non-woven fabric strip 13, a second multi-overlapped non-woven fabric strip 15, a third multi-overlapped non-woven fabric strip 17, and a fourth multi-overlapped non-woven fabric strip 19. Each fabric strip 13, 15, 17, 19 is spirally wound, such as wrapped about an axis or coiled, or more preferably helically wound in overlapping layers to form overlapping bands 14, 16, 18, 20, respectively. While a helical wind is shown, other spiral arrangements may be used. The radially interior surface 21 of band 14 forms the periphery of an axially extending annular space that extends from one end 25 of the filter element to the oppositely facing end 27 of the filter media 11. In the drawings, the thickness of the fabric is exaggerated.

In FIG. 7 of the drawings, the numeral 47 designates a hollow cylindrical mandrel with an annular exterior surface 49 and an annular interior surface 51, the annular interior surface 51 forming the periphery of a cylindrical channel 53, through which flows a liquid or gas heat exchange medium (not shown). Band 14 of multi-overlapped non-woven fabric strip 13 is shown overlapped by band 16 of multi-overlapped non-woven fabric strip 15, which in turn is overlapped by band 18 of multi-overlapped non-woven fabric strip 17, which is then overlapped by band 20 of multi-overlapped non-woven fabric strip 19.

In another embodiment, multi-overlapped coreless filter media 11 of the present invention is circumscribed by an annular seal holder 85, as described in U.S. Pat. No. 6,168,647, and depicted in FIG. 8. Referring to FIG. 8, seal holder 85 is preferably made of polyester and is permanently sealed, or affixed, to a filter wall 81. Seal holder 85 is sealingly bonded to filter wall 81 by a heat treatment, but it should be understood that seal holder 85 may be sealed to filter wall by other conventional means, such as glue or adhesive. It is preferable that seal holder 85 does not compress the layers of filter element 11. Seal holder releasably carries an annular seal 87, preferably a chevron-type seal, as will be explained in more detail below.

Seal holder 85 and seal 87 separate filter media 11 into two portions: an inlet portion 89a and an outlet portion 89b. It is not necessary that inlet portion 89a and outlet portion 89b are of the same length. Indeed, depending upon the application, it may be necessary to offset seal holder 85 and seal 87 from the axial center of filter media 11. It is important to note that both inlet portion 89b and outlet portion 89b are of generally homogenous construction and thus integral and continuous; therefore, inlet portion 89a and outlet portion 89b are functionally identical, although the lengths of inlet portion 89a and 89b may vary. When seal 87 is a chevron-type seal, inlet portion 89a and outlet portion 89b are determined by the orientation of seal 87, as will be explained in more detail below. On the other hand, if seal 87 is an a-ring, or some other type of seal whose functionality is independent of flow direction, then inlet portion 89a and outlet portion 89b may be interchangeable. It should be understood that due to differences in the sealing characteristics between a chevron type seal and an a-ring type seal, the two seals may not be interchangeable for a given filter media 11.

Inlet portion 89a terminates with a filter inlet cap 91a, and outlet portion 89b terminates with a filter outlet cap 91b. It is preferable that both filter inlet cap 91a and filter outlet cap 91b are identical, but for reasons explained below, filter inlet cap 91a and filter outlet cap 91b may be of varying configurations. Filter inlet cap 91a and filter outlet cap 91b form a fluid-tight seal with filter media 11 such that all fluids in the gas stream must pass through filter wall 81. Filter inlet cap 91a has a filter inlet cap post 93a that protrudes longitudinally outward from filter element 11. Filter inlet cap post 93a preferably tapers inwardly at its outermost extent. In a similar fashion, filter outlet cap 91b has a filter outlet cap post 93b that protrudes longitudinally outward from filter media 11. Filter outlet cap post 93b preferably tapers inwardly at its outermost extent. Filter inlet cap 91a and filter outlet cap 91b are illustrated having a filter inlet cap flange 95a and a filter outlet cap flange 95b, respectively, although filter inlet cap 91a and filter outlet cap 91b may also be flush with filter wall 81.

Referring to FIG. 9, a blow-up view of III of FIG. 8 is illustrated. As mentioned above, inlet portion 89a and outlet portion 89b are functionally identical. When seal 87 is a chevron-type seal, as is preferable, the orientation of seal 87 determines which portion of filter media 11 represents inlet portion 89a, and which portion of filter media 11 represents outlet portion 89b. Although the orientation of chevron-type seal 87 determines which portion of filter media 11 represents inlet portion 89a, it should be understood that other means of ensuring proper installation of filter media 11 exist. For example, filter inlet cap post 93a and filter inlet cap post 93b may be of different sizes or shapes, or filter inlet cap flange 95a and filter outlet cap flange 95b may be of different sizes or shapes.

Referring now to FIG. 10 in the drawings, seal holder 85 is generally U-shaped, having a seal channel 101 and generally parallel legs 103a and 103b. Seal channel 101 is adapted to receive and carry seal 87. Legs 103a and 103b are preferably of the same length, but may be of varying lengths depending upon the type of seal 87 carried by seal holder 85. Seal 87 is preferably a chevron-type seal made of an elastomer, but may be other types of seals, such as a conventional O-ring made out of other suitable materials. Preferably, seal 87 is releasably sealed and carried in seal channel 101 by a tension fit, but it should be understood that seal 87 may be bonded or otherwise adhered in seal channel 101, or to legs 103a or 103b of seal holder 85.

When seal 87 is a chevron-type seal, seal 87 includes a seal base portion 105, a seal vertex portion 107, and a seal cone portion 109. Seal base portion 105 and seal cone portion 107 are integrally joined together at seal vertex portion 107. Seal cone portion 109 is preferably frusto-conical-shaped, having a small-diameter end 111, and a large-diameter end 113. It is preferable that seal base portion 105 and seal cone portion 109 form an angle α of about 60°.

It is reiterated that the preferred filter media employed in the present invention, as described above, is provided with a surface area that includes multiple overlapping layers of media (i.e., bands) whereby adjacent layers have an intersection plane at the point of joining. Such a design, in an embodiment, can enhance the filtration capacity of the bands. Moreover, with such a design, a gradient of density within the filter media 11 can be provided across the depth of the filter media 11.

With reference to another embodiment of the present invention, to further enhance the filtration capacity of filter media 11, the present invention may provide filter media 11 with an interlay of media within at least one of bands 14, 16, 18, 20, as disclosed in U.S. Pat. No. 8,062,523. The presence of such an interlay in filter media 11 can, in an embodiment, provide filter media 11 with additional surface area for filtration. In particular, to the extent that the interlay may be different in characteristics and properties from the underlying filter element bands 14, 16, 18, 20, there can be a distinct and abrupt change in density, fiber size, etc., that, in effect, create additional surface area within the contiguous construction of a filter element of the present invention. This interlay can also create the ability to change direction of flow and to increase the deposition of specifically sized contaminants.

Looking now at FIG. 11A, there is illustrated a cross-sectional view of a multi-overlapped coreless filter media 60, in accordance with one embodiment of the present invention. Similar to filter media 11, filter media 60 can include multiple bands 61, 62, 63 and 64. Of course, additional or fewer bands may be provided should that be desired. Filter element 60 can further include an interlay 65 disposed within at least one overlapping band, such as band 61. The presence of interlay 65 within overlapping band 61 of filter media 60 can allow the filter media 60 to be designed in such a way as to control and impart a particular filtration or flow pattern of the fluid moving within filter media 60, for instance, in a substantially axially direction.

In accordance with an embodiment of the present invention, interlay 65 may be made from a material or materials that can provide characteristics different from those of the bands 61 to 64. In one embodiment, these characteristics may be imparted based on the size of, for instance, the fibers, as well as the process or recipe used in making the interlay 65. In general, the fibers used can come in different diameters. In an embodiment, the interlay 65 can be made up from a mixture of fibers of widely different diameters. This mixture or recipe can determine the performance or characteristics of the interlay 65, and depending on the application, the performance or characteristics of interlay 65 can be substantially different or slightly different than the characteristics or performance of bands 61 to 64.

Examples of materials (thermoplastic and natural) that can be used in the manufacture of interlay 65 can vary widely including metals, such as stainless steel, inorganic components, like fiberglass or ceramic, organic cellulose, paper, or organic polymers, such as polypropylene, polyester, nylon, etc., or a combination thereof. These materials have different chemical resistance and other properties.

In addition, looking now at FIG. 11B, interlay 65, in one embodiment, may be provided from a strip, such as strip 651, with a width substantially similar in size to that of a strip, such as strip 611, being used in making the band within which the interlay 65 is disposed. Alternatively, the interlay 65 may be provided from a strip with a width measurably less than the width of the strip used in the band within which the interlay 65 is disposed. In an embodiment, the interlay 65 may include a width approximately 2 inches less than the width of the strip used in the band.

To dispose the interlay 65 in the manner illustrated in FIG. 11A, at the beginning of the manufacturing process, strip 651 from which interlay 65 is formed may be placed substantially parallel to and against a surface of, for example, strip 611 used in the formation of, for instance, band 61. Strip 611, manufactured by the process indicated above, can be non-woven in nature. In an embodiment, the strip 651, which can also be non-woven or otherwise, may be placed against a surface of strip 611 that subsequently can become an inner surface of band 61. Alternatively, strip 651 may be placed against a surface of strip 611 that subsequently can become an outer surface of band 61. Thereafter, as strip 611 is wound about mandrel 47 to form band 61, the strip 651 can be wound simultaneously along with strip 611 of band 61 to provide the configuration shown in FIG. 11A. In other words, for example, each layer of the interlaying strip 651 may be sandwiched between two adjacent overlapping layers of the non-woven strip 611. It should be noted that the interlay 65 within band 61 is provided above and below pathway 67 formed by the mandrel 47 during the winding process, such as that illustrated in FIG. 11A. Moreover, despite being illustrated in connection only with band 61, it should be appreciated that interlay 65 may be disposed within one or more of the remaining bands 62 to 64. Furthermore, each interlay 65 in each of bands 61 to 64, in an embodiment, may be provided with different or similar characteristics to the other interlays, depending on the particular application or performance desired.

In an alternate embodiment, as illustrated in FIG. 12, instead of providing interlay 65 within overlapping band 61, an interleaf 75 may provide circumferentially about overlapping band 71. To dispose the interleaf 75 in the manner illustrated in FIG. 12, in one embodiment, subsequent to the formation of overlapping band 71, a strip, used in the formation of interleaf 75, may be wrapped or wound in an overlapping manner similar to that for band 71 about an exterior surface of band 71 to provide an overlapping profile exhibited by interleaf 75 in FIG. 12. Of course, although illustrated with only one interleaf, interleaf 75 may be provided about one or more of the remaining bands in filter media 70.

Alternatively, rather than providing an overlapping interleaf 75, an interleaf 85, looking now at FIG. 13, may be disposed as one layer along an entire length of filter media 80 and within band 81. In this embodiment, strip 851 may be provided with a length substantially similar to that of filter media 80 and a width substantially similar to a circumference of band 81. That way, band 81 of filter media 80 may be positioned along the length of strip 851 and the width of strip 851 subsequently wrapped once about band 81. This, of course, can be done during the formation of band 81, so that interleaf 85 may be provided within band 81, or after the formation of band 81, so that interleaf 85 may be provided about an exterior surface of band 81. Interleaf 85 may also be provided about one or more of the remaining bands in filter media 80.

In a related embodiment, strip 851 may be provided with a length shorter than that of filter media 80. With a shorter length, interleaf 85 may be provided about each band of filter media 80 and in a staggered manner from one band to the next (not shown).

In addition to the materials (e.g., types and sizes), the characteristics or properties of the interlay 65 as well as bands 61 to 64, which may be referred to hereinafter as media, can be dependent on pore size, permeability, basis weight, and porosity (void volume) among others. The combination of these properties can provide the interlay 65, along with bands 61 to 64, with a particular flow capacity (differential pressure of fluid across the filter), micron rating (the size of the particles that will be removed from the filter media 60, particle holding capacity (the amount of contaminant that can be removed from the process by the filter media 60 before it becomes plugged), and physico-chemical properties.

Moreover, by providing filter media 60 with interlay 65 having different characteristics and properties from those exhibited by the multiple overlapping bands 61 to 64, there can be, for example, a distinct and abrupt change in density within the filter element 60 that, in effect, can create additional surface area, thereby allowing for the generation of a gradient density within filter media 60 at a micro level as well as a macro level.

The presence of interlay 65 within filter element 60 can also impart, in an embodiment, a substantially axial fluid flow pathway along the filter element 60. Generally, the flow of fluid through the overlapping bands, for example, bands 61 to 64, is in a substantial radial direction across filter media 60 either from outside to inside or from inside to outside. However, using an interlay of more dense or less permeable media, as described above, the flow of the fluid across filter element 60 can be directed substantially axially along the length of the filter media 60, as illustrated by arrow 66 in FIG. 11A.

All references, including publications, patent applications, and patents cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A low surface energy filter element comprising a synthetic non-woven media comprising at least one hydrophobic layer, the at least one hydrophobic layer has a water contact angle of greater than 120° when the media is immersed in Jet-A fuel.

2. A low surface energy filter element according to claim 1 wherein the hydrophobic layer is superhydrophic.

3. A low surface energy filter element according to claim 1 wherein the non-woven media is multi-layered.

4. A low surface energy filter element according to claim 1 wherein the hydrophobic layer is made from nanofiber having an average diameter of less than 800 nanometers.

5. A low surface energy filter element according to claim 4 wherein the nanofiber is selected from the group consisting of a nylon, a polyvinylidene fluoride (PVDF), a polyurethane (PU), a polyacrylonitrile (PAN), a cellulose Tri Acetate (CTA), a polymethylmethacrylate (PMMA), a poly(vinylidene fluoride-co-hexafluoropropene) (PVDF-HFP), a poly(4-methyl-1-pentene) (PFMOP) and a polytetrafluoroethylene (PTFE).

6. A low surface energy filter element according to claim 4 wherein the nanofiber is coated with fluoropolymer.

7. A low surface energy filter element according to claim 6 wherein the nanofiber is a nylon.

8. A low surface energy filter element according to claim 1 wherein the non-woven media comprises two hydrophobic layers.

9. A low surface energy filter element according to claim 8 wherein the two hydrophobic layers are a fluorocarbon coated thermoplastic resin and a fluoropolymer non-woven media.

10. A low surface energy filter element according to claim 9 wherein the fluoropolymer non-woven media is selected from the group consisting of ethylene chlorotrifluoroethylene and polyvinylidene fluoride.

11. A low surface energy filter element according to claim 10 wherein the two hydrophobic layers are bonded to each other to form a helical wound tube.

12. A low surface energy filter element according to claim 11 wherein the polyethylene terephthalate never reaches the outside surface of the helical wound tube.

13. A low surface energy filter element according to claim 1 wherein the non-woven media comprises a first hydrophobic layer and a second hydrophobic layer; the first hydrophobic layer spirally wound upon itself in multiple overlapping layers to form a band of a selected radial thickness.

14. A low surface energy filter element according to claim 13 wherein the second hydrophobic layer is an interlaying layer being disposed in a spirally wound manner, so as to provide adjacently overlapping layers within the band formed by the first hydrophobic layer.

15. A low surface energy filter element according to claim 14 wherein the first hydrophobic layer is a thermoplastic resin.

16. A low surface energy filter element according to claim 15 wherein the second hydrophobic layer is selected from the group consisting of ethylene chlorotrifluoroethylene, PVDF, polystyrene, plasma coated PEM and plasma coated nanofiber.

17. A low surface energy filter element according to claim 10 wherein the thermoplastic resin is selected from the group consisting of polyester and polypropylene.

18. A low surface energy filter element according to claim 17 wherein the thermoplastic resin is a polyester.

19. A low surface energy filter element according to claim 18 wherein the polyester is polyethylene terephthalate.

20. A low surface energy filter element according to claim 16 wherein the thermoplastic resin is selected from the group consisting of polyester and polypropylene.

21. A low surface energy filter element according to claim 20 wherein the thermoplastic resin is a polyester.

22. A low surface energy filter element according to claim 21 wherein the polyester is polyethylene terephthalate.

23. The low surface energy filter element of claim 1, wherein the hydrophobic layer has an average pore size of between 30 and 180 micron excluding nanofibers if carried by the hydrophobic layer.

24. The low surface energy filter element of claim 23, wherein the minimum pore size is about 15 micron.

25. The low surface energy filter element of claim 1, wherein the hydrophobic layer has an average pore size of between 0.50 and 1.00 micron.

26. The low surface energy filter element of claim 25, wherein the hydrophobic layer has a minimum pore size of about 0.25 micron and a maximum pore size of about 1.50 micron.

27. The low surface energy filter element of claim 1, wherein the hydrophobic layer comprises fibers with terminating ends of at least some of the fibers freely projecting generally in a cantilever manner from the upstream surface of the media, which when stretched straight measure greater than 3 millimeters.

28. A method of filtering using the low surface energy filter element of claim 1, comprising:
arranging the low surface energy filter element in a continuous phase liquid comprising a hydrocarbon liquid stream; separating a dispersed liquid phase comprising water from the hydrocarbon liquid stream with the low surface energy filter element.

29. The method of claim 28, wherein the hydrocarbon liquid stream is a fuel.

30. The filter element of claim 1, wherein the non-woven media has a total thickness of at least ¼ inch.

31. The filter element of claim 1, wherein the non-woven media has a total thickness of at least ½ inch.
